(19) United States
(12) Patent Application Publication
(10) Pub. No.: US 2025/0152137 A1
(43) Pub. Date: May 15, 2025

(54) ULTRASONIC TRANSDUCER DEVICE AND ULTRASONIC PROBE USING THE SAME

(71) Applicant: Qisda Corporation, Taoyuan City (TW)

(72) Inventor: Fu-Sheng JIANG, Taoyuan City (TW)

(73) Assignee: Qisda Corporation, Taoyuan City (TW)

(21) Appl. No.: 18/938,378

(22) Filed: Nov. 6, 2024

(30) Foreign Application Priority Data
Nov. 13, 2023 (TW) 112143609

(51) Int. Cl. A61B 8/00; B06B 1/02

(52) U.S. Cl. CPC A61B 8/4494; B06B 1/0292; B06B 2201/76

(57) ABSTRACT

An ultrasonic transducer device includes a substrate, an ultrasonic oscillation unit, a ground signal line, a DC voltage signal line and an AC voltage signal line. The substrate includes an active component area and a wiring area located around the active component area. The ultrasonic oscillation unit includes a first electrode layer, a second electrode layer, a third electrode layer and a cavity. The first electrode layer is located on a first side of the cavity, and the second electrode layer and the third electrode layer are located on a second side of the cavity. The ground signal line, disposed in the wiring area, is electrically connected to the first electrode layer. The DC voltage signal line, disposed in the wiring area, is electrically connected to the second electrode layer. The AC voltage signal line, disposed in the wiring area, is electrically connected to the third electrode layer.

This application claims the benefit of Taiwan application Serial No. 112143609, filed Nov. 13, 2023, the subject matter of which is incorporated herein by reference.

### BACKGROUND OF THE INVENTION

### Field of the Invention

The invention relates in general to an ultrasonic transducer device and an ultrasonic probe using the same.

### Description of the Related Art

Along with the advance in medical technology, ultrasonic detection technology is also getting more and more matured. Generally speaking, the probe used in ultrasonic detection emits ultrasonic signals to the underneath of the skin. Moreover, for medical purposes, the probe can detect the shape and position of an object under the skin and invisible to the naked eye according to the reflection of the ultrasonic signals.

To improve the ultrasonic signal and imaging quality, the peripheral circuit of the ultrasonic transducer device must be modified so that the drive circuit can be integrated to the peripheral circuit of the ultrasonic transducer device.

### SUMMARY OF THE INVENTION

The invention is directed to an ultrasonic transducer device and an ultrasonic probe using the same capable of increasing the performance of the ultrasonic transducer device and reducing the volume of the device.

According to one embodiment of the present invention, an ultrasonic transducer device is provided. The ultrasonic transducer device includes a substrate, an ultrasonic oscillation unit, a ground signal line, a direct current (DC) voltage signal line and an alternate current (AC) voltage signal line. The substrate includes an active component area and a wiring area located around the active component area. The ultrasonic oscillation unit is disposed in the active component area and includes a first electrode layer, a second electrode layer, a third electrode layer and a cavity. The first electrode layer is located on a first side of the cavity, and the second electrode layer and the third electrode layer are located on a second side of the cavity. The second side is opposite to the first side. The ground signal line is disposed in the wiring area and is electrically connected to the first electrode layer. The DC voltage signal line is disposed in the wiring area and is electrically connected to the second electrode layer. The AC voltage signal line is disposed in the wiring area and is electrically connected to the third electrode layer. The ground signal line and the DC voltage signal line have electrical connection areas of different levels in the wiring area.

According to another embodiment of the present invention, an ultrasonic probe is provided. The ultrasonic probe includes a hand-held casing and an ultrasonic transducer device. The hand-held casing has a first end and a second end. The ultrasonic transducer device is disposed at the first end or the second end of the hand-held casing. The ultrasonic transducer device includes a substrate, an ultrasonic oscillation unit, a ground signal line, a DC voltage signal line and an AC voltage signal line. The substrate includes an active component area and a wiring area located around the active component area. The ultrasonic oscillation unit is disposed in the active component area and includes a first electrode layer, a second electrode layer, a third electrode layer and a cavity. The first electrode layer is located on a first side of the cavity. The second electrode layer and the third electrode layer are located on a second side of the cavity. The first side is opposite to the second side. The ground signal line is disposed in the wiring area and is electrically connected to the first electrode layer. The DC voltage signal line is disposed in the wiring area and is electrically connected to the second electrode layer. The AC voltage signal line is disposed in the wiring area and is electrically connected to the third electrode layer. The second electrode layer and the third electrode layer have electrical connection areas of identical or different levels in the active component area.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIGS. 1A and 1B respectively are cross-sectional views of an ultrasonic transducer device according to an embodiment of the invention.

FIG. 2A is a top view of an ultrasonic transducer device according to an embodiment of the invention.

FIG. 2B is a top view of an ultrasonic transducer device according to another embodiment of the invention.

FIG. 2C is a top view of an ultrasonic transducer device according to another embodiment of the invention.

FIGS. 2D and 2E respectively are top views of an ultrasonic transducer device according to another embodiment of the invention.

FIG. 3A is a top view of an ultrasonic transducer device according to an embodiment of the invention.

FIG. 3B is a top view of an ultrasonic transducer device according to another embodiment of the invention.

FIG. 4 is a cross-sectional view of an ultrasonic transducer device according to another embodiment of the invention.

FIGS. 5A-5D respectively are schematic diagrams of an ultrasonic transducer device according to another embodiment of the invention.

FIG. 6 is a top view of an ultrasonic probe according to an embodiment of the invention.

### DETAILED DESCRIPTION OF THE INVENTION

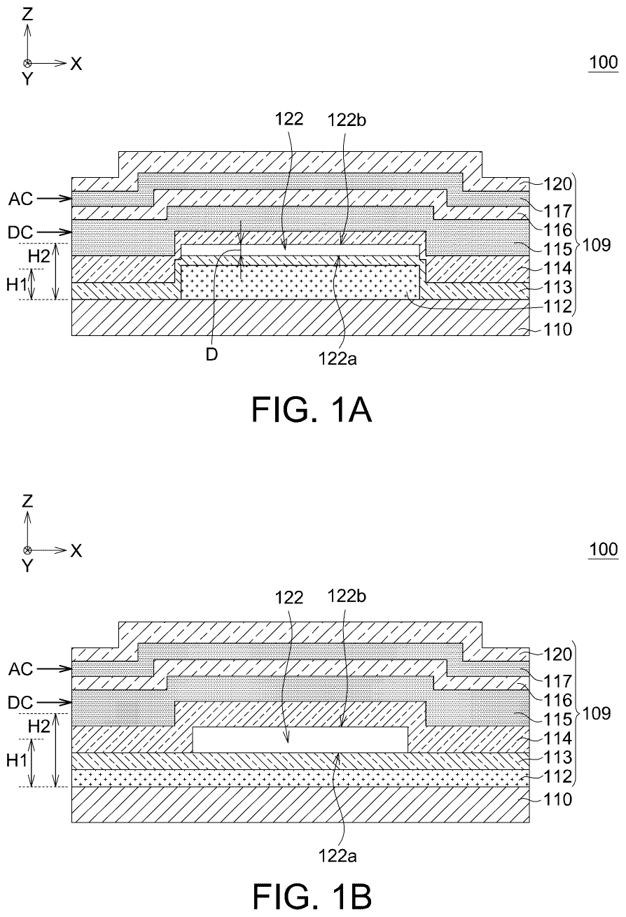

Referring to FIGS. 1A and 1B, cross-sectional views of an ultrasonic transducer device 100 according to an embodiment of the invention are respectively shown. Only one ultrasonic oscillation unit 109 is illustrated in FIGS. 1A and 1B. In the present embodiment, the ultrasonic transducer device 100 is formed of a plurality of capacitive micro-machined ultrasonic transducers (CMUT) and includes a substrate 110 and an ultrasonic oscillation unit 109. The ultrasonic oscillation unit 109 is disposed on the substrate 110 and includes a first electrode layer 112, a first insulation layer 113, a second insulation layer 114, a second electrode layer 115, a third insulation layer 116, a third electrode layer 117, and a cavity 122 located between the first insulation layer 113 and the second insulation layer 114. Besides, the ultrasonic oscillation unit 109 is also referred as ultrasonic oscillator.

In an embodiment, the first electrode layer 112 and the first insulation layer 113 are located on a first side 122a of the cavity 122 (such as lower side), the second insulation layer 114, the second electrode layer 115, the third insulation layer 116 and the third electrode layer 117 are located on a second side 122b of the cavity 122 (such as upper side), and the first side 122a is opposite to the second side 122b. The first electrode layer 112 can be used to receive a ground voltage, the second electrode layer 115 can be used to receive a direct current voltage DC, and the third electrode layer 117 can be used to receive an alternate current voltage AC. Or, in another embodiment, the second electrode layer 115 can be used to receive an alternate current voltage AC, and the third electrode layer 117 can be used to receive a direct current voltage DC. For instance, the second electrode layer 115, relative to the first electrode layer 112, is driven by a direct current voltage DC to recess towards the first electrode layer 112, making the first insulation layer 113 closer to the second insulation layer 114. Or, the third electrode layer 117, relative to the first electrode layer 112, is driven by an alternate current voltage AC or an external acoustic pressure to generate vibrations and accordingly generate ultrasonic signals. The pulse period of the alternate current voltage AC can determine the vibration frequency of the outputted ultrasonic signal and generate the ultrasonic signals of different frequencies.

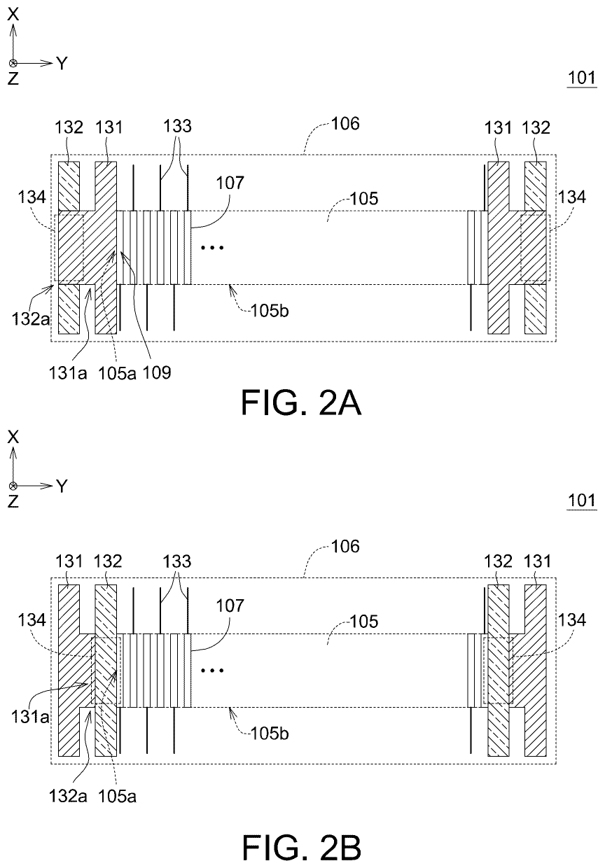

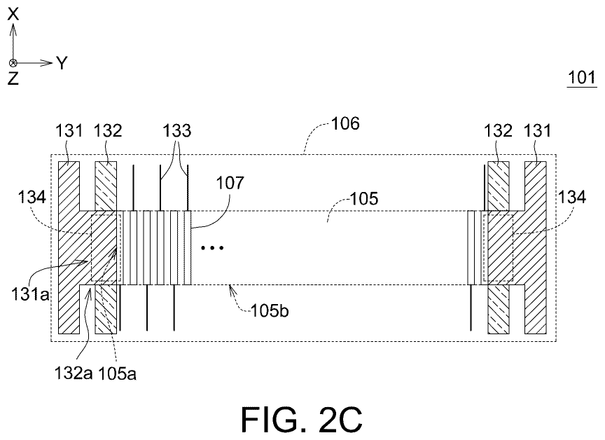

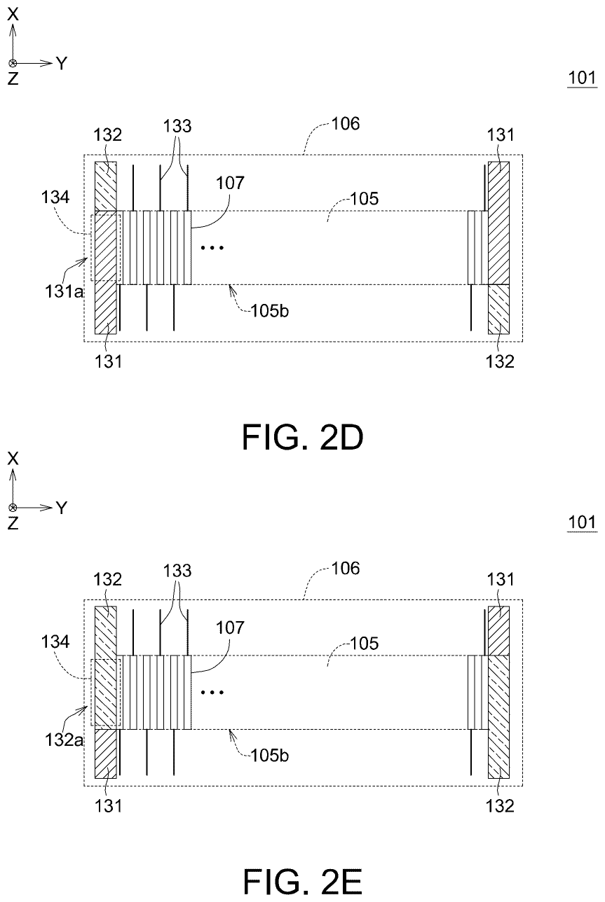

Although only one ultrasonic oscillation unit 109 is illustrated in FIGS. 1A and 1B, it can be understood that the ultrasonic oscillation units 109 are one-dimensionally or two-dimensionally arranged to form a transducer unit 107. For instance, the transducer unit 107 is formed of a plurality of ultrasonic oscillation units 109 which are extended along the X-axis direction, electrically connected to each other, and arranged as a one-dimensional array; or the transducer unit 107 is formed of a plurality of ultrasonic oscillation units 109 which are extended along a first direction (the X-axis) and a second direction (the Y-axis) perpendicular to the first direction, electrically connected to each other, and arranged as a two-dimensional matrix. Refer to FIG. 2A. A transducer unit 107 is formed of a plurality of ultrasonic oscillation units 109 arranged as a one-dimensional array along the X-axis direction. Each transducer unit 107 can separately receive an alternate current voltage AC signal and vibrate or receive a direct current voltage DC signal and maintain film layer at the recess position. Besides, each transducer unit 107 can separately receive an external acoustic pressure signal (such as an ultrasonic signal) and vibrate.

Refer to FIGS. 1A and 1B. The substrate 110 can be implemented by a semiconductor wafer or a glass substrate. The first electrode layer 112, a first insulation layer 113, a second insulation layer 114, the second electrode layer 115, a third insulation layer 116, and a third electrode layer 117 are formed on the substrate 110 by using a microelectromechanical process or a semiconductor process. The semiconductor process includes a thin film deposition process, a lithography process, an etching process and a cleaning process. The thin film deposition process includes subsequently depositing a plurality of film layers on the substrate 110. The lithography process includes defining the pattern and shape of each film layer through a photoresist layer, so that each film layer can be stacked according to a predetermined sequence. The etching process includes removing redundant parts of a film layer to form a cavity 122 in the film layer or to form a groove. The cleaning process includes removing a photoresist layer, an etching liquid and other solvents.

In an embodiment, the first electrode layer 112, the first insulation layer 113, the second insulation layer 114, the second electrode layer 115, the third insulation layer 116, and the third electrode layer 117 are sequentially stacked in a bottom-up manner. The first electrode layer 112 is formed on the substrate 110. The first insulation layer 113 covers the first electrode layer 112. The second insulation layer 114 covers the first insulation layer 113. A cavity 122 is formed between the first insulation layer 113 and the second insulation layer 114, so that the first insulation layer 113 and the second insulation layer 114 are separated by an interval D at the cavity 122. The formation of the cavity 122 is as follows: a sacrificial layer is formed between the first insulation layer 113 and the second insulation layer 114 in advance, then, the sacrificial layer is etched with an etching liquid, so that a cavity 122 is formed between the first insulation layer 113 and the second insulation layer 114. Afterwards, the second electrode layer 115 is formed on the second insulation layer 114. Then, the third insulation layer 116 covers the second electrode layer 115, and the third electrode layer 117 covers the third insulation layer 116. In an embodiment, the ultrasonic oscillation unit 109 further includes an upper insulation layer 120 disposed on the second electrode layer 115 and used for protecting the second electrode layer 115. The upper insulation layer 120, the first insulation layer 113 and the second insulation layer 114 can be formed of identical or different materials. The insulation layer disclosed above can be formed of silicon oxide (such as SiO₂), nitride (such as SiN), nitrogen oxide or any suitable dielectric insulating materials. Refer to FIG. 1B. In the above embodiment, the first electrode layers 112 of the ultrasonic oscillation units 109 can be serially connected during the manufacturing of the first electrode layers 112 for the convenience of the manufacturing process or for the purpose of electrical performance.

Refer to FIGS. 1A, 1B and FIGS. 2A-2E. FIGS. 2A-2E respectively are top views of an ultrasonic transducer device 101 according to an embodiment of the invention. The substrate has an active component area 105 and a wiring area 106 located around the active component area 105. The active component area 105 includes a plurality of ultrasonic oscillation units 109, which are arranged as a one-dimensional array in the X-axis direction to form a transducer unit 107, and the transducer units 107 are arranged in parallel along the Y-axis direction. Besides, the wiring area 106 includes a ground signal line 131, a DC voltage signal line 132 and an AC voltage signal line 133, wherein the ground signal line 131 is electrically connected to the first electrode layer 112 of each of the ultrasonic oscillation units 109 (referring to FIG. 1), the DC voltage signal line 132 is electrically connected to the second electrode layer 115 of each of the ultrasonic oscillation units 109 (referring to FIG. 1), and the AC voltage signal line 133 is electrically connected to the third electrode layer 117 of each of the ultrasonic oscillation units 109 of the transducer unit 107 (referring to FIG. 1).

As indicated in FIG. 2A-2C, the active component area 105 has a first side connection area 105a and a second side connection area 105b. The first side connection area 105a is different from the second side connection area 105b. The ground signal line 131 and the DC voltage signal line 132 are disposed in the first side connection area 105a, and the AC voltage signal line 133 is disposed in the second side connection area 105b. In FIG. 2A-2C, the first side connection area 105a is located on any side or two opposite sides of the active component area 105, and the second side connection area 105b is located on any side or two opposite sides of the active component area 105. For instance, two ground signal lines 131 and two DC voltage signal lines 132 located on the two sides are respectively electrically connected to at least a corresponding part of the ultrasonic oscillation units 109. Also, the AC voltage signal lines 133 located on the other two sides are respectively electrically connected to at least a corresponding part of the ultrasonic oscillation units 109. The input of the AC voltage signal lines 133 can be one-directional or multi-directional, and the AC voltage signal lines 133 can be arranged in a staggered or aligned manner.

Besides, in FIG. 2A-2C, the ground signal line 131 and the DC voltage signal line 132 have electrical connection areas of different levels in the wiring area 106. For instance, viewing from the top, the overlapping area 134 of the ground signal line 131 and the DC voltage signal line 132, in response to electrical isolation, have different levels in the wiring area 106. Besides, in the non-overlapping area, the ground signal line 131 and the DC voltage signal line 132 can have different or identical levels in the wiring area 106 as long as the ground signal line 131 and the DC voltage signal line 132 are electrical isolated.

In FIGS. 2A and 2C, the ground signal line 131 and the DC voltage signal line 132 are disposed on any side or two opposite sides of the wiring area 106 along the Y-axis direction, and the projections of the electrical connection area of the ground signal line 131 and the electrical connection area of the DC voltage signal line 132 in the vertical direction (Z axis) at least have an overlapping area 134. For instance, the electrical connection area 132a of the DC voltage signal line 132 has a first level H1 (illustrated in FIG. 1), the electrical connection area 131a of the ground signal line 131 has a second level H2 (illustrated in FIG. 1), and the second level H2 is higher than the first level H1, so that the electrical connection area 131a of the ground signal line 131 can cross over the electrical connection area 132a of the DC voltage signal line 132 and is electrically isolated from the electrical connection area 132a of the DC voltage signal line 132 by the first insulation layer 113. Besides, the AC voltage signal line 133 is disposed on any side or two opposite sides of the wiring area 106 along the Y-axis direction perpendicular to the X-axis direction. In an embodiment, the AC voltage signal line 133 does not overlap the projections of the ground signal line 131 and the DC voltage signal line 132 in the vertical direction (Z axis).

In FIG. 2B, the ground signal line 131 and the DC voltage signal line 132 are disposed on any side or two opposite sides of the wiring area 106 along the Y-axis direction, and the projections of the electrical connection area 131a of the ground signal line 131 and the electrical connection area 132a of the DC voltage signal line 132 in the vertical direction (Z axis) at least have an overlapping area 134. For instance, the electrical connection area 131a of the ground signal line 131 has a first level H1, the electrical connection area 132a of the DC voltage signal line 132 has a second level H2, and the second level H2 is higher than the first level H1, so that the electrical connection area 132a of the DC voltage signal line 132 can cross over the electrical connection area 131a of the ground signal line 131 and is isolated from the electrical connection area 131a of the ground signal line 131 by the first insulation layer 113. Besides, the AC voltage signal line 133 is disposed on any side or two opposite sides of the wiring area 106 along the X-axis direction. In an embodiment, the AC voltage signal line 133 does not overlap the projections of the ground signal line 131 and the DC voltage signal line 132 in the vertical direction (Z axis).

Refer to FIG. 2D. The electrical connection area 131a of the ground signal line 131 is higher than the electrical connection area 132a of the DC voltage signal line 132 in the overlapping area 134, and the extending direction of the ground signal line 131 is opposite to that of the DC voltage signal line 132 (along the X-axis direction). Refer FIG. 2E. The electrical connection area 132a of the DC voltage signal line 132 is higher than the electrical connection area 131a of the ground signal line 131 in the overlapping area 134, and the extending direction of the DC voltage signal line 132 (along the X-axis direction) is opposite to that of the ground signal line 131. However, the extending direction of the DC voltage signal line 132 can be identical to that of the ground signal line 131, and the invention does not have specific restrictions regarding such arrangement.

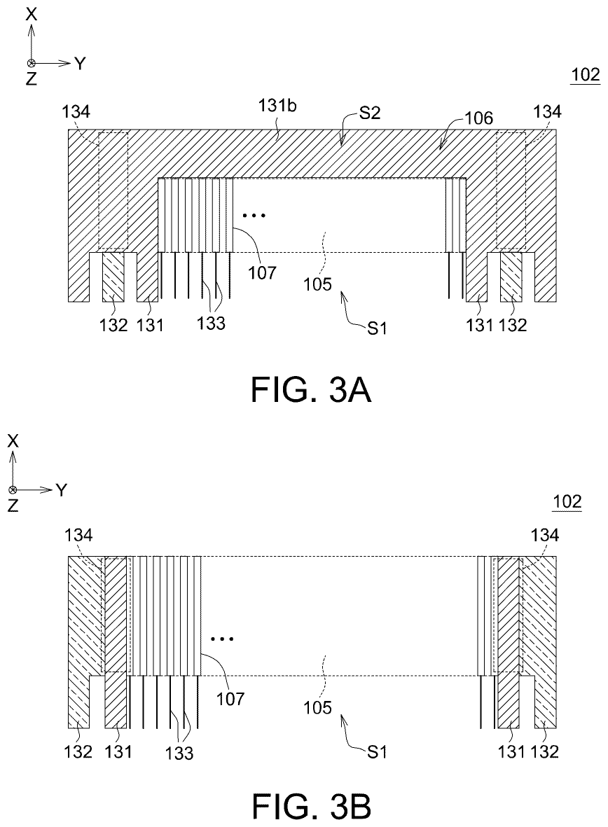

Referring to FIGS. 3A and 3B, top views of an ultrasonic transducer device 102 according to an embodiment of the invention are respectively shown. The embodiment of FIGS. 3A is different from that of FIG. 3B in that the AC voltage signal line 133 is disposed on the first side S1 of the wiring area 106 along the X-axis direction, that is, the AC voltage signal line 133 is disposed on only one side. Besides, in FIG. 3A, the ground signal line 131 is disposed on not only two opposite sides of the wiring area 106 along the Y-axis direction but also includes an electrical connection area 131b located on a second side S2 of the wiring area 106 along the X-axis direction. The first side S1 is opposite to the second side S2. The ground signal line 131 can be extended out from the first side S1, and so can the DC voltage signal line 132 be extended out from the first side S1. In FIG. 3B, each of the AC voltage signal line 133, the ground signal line 131 and the DC voltage signal line 132 can be coiled out from one single side (the first side S1). In another embodiment, each of the AC voltage signal line 133, the ground signal line 131 and the DC voltage signal line 132 can be extended out from two sides (the first side S1 and the second side S2) to be applicable to various circuit designs required by the ultrasonic transducer device 102.

The ground signal line 131 of an embodiment and the first electrode layer 122 of FIG. 1 can be made of identical material and concurrently formed on the substrate 110, and required patterns can be respectively formed through the use of a patterned film layer. The ground signal line 131 and the first electrode layer 122 have different levels and can be electrically connected through conductive vias. Similarly, the DC voltage signal line 132 and the second electrode layer 115 have different levels and can be electrically connected through conductive vias.

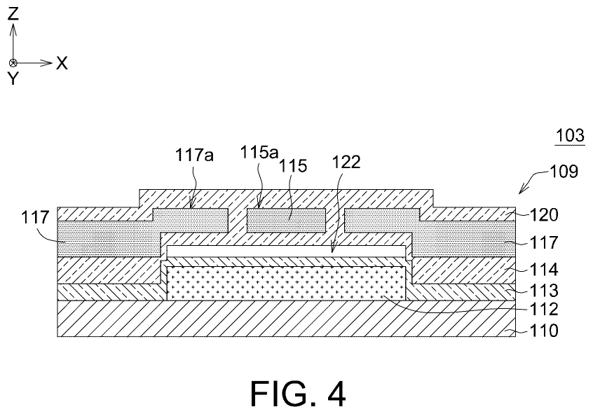

Besides, in FIG. 1, the first electrode layer 112 has an electrical connection area of identical level in the active component area 105, the second electrode layer 115 has an electrical connection area of identical level in the active component area 105, and the third electrode layer 117 has an electrical connection area of different levels in the active component area 105. Besides, the second electrode layer 115 and the third electrode layer 117 have electrical connection areas of different levels in the active component area 105. In another embodiment, the second electrode layer 115 and the third electrode layer 117 can have electrical connection areas of identical level in the active component area 105 as indicated in FIG. 4.

Referring to FIG. 4, a cross-sectional view of an ultrasonic transducer device 103 according to another embodiment of the invention is shown. In the present embodiment, the ultrasonic oscillation unit 109 includes a first electrode layer 112, a first insulation layer 113, a second insulation layer 114, a second electrode layer 115, a third electrode layer 117, and a cavity 122 located between the first insulation layer 113 and the second insulation layer 114. The second electrode layer 115 and the third electrode layer 117 are formed on the second insulation layer 114, and have electrical connection areas 115a and 117a of identical levels in the active component area 105 (referring to FIG. 2A). The second electrode layer 115 and the third electrode layer 117 are electrically isolated from each other, wherein the second electrode layer 115 is located in the center area, and the third electrode layer 117 is located in the peripheral area of the second electrode layer 115; or, the third electrode layer 117 is located in the center area, and the second electrode layer 115 is located in the peripheral area of the third electrode layer 117. In an embodiment, the third electrode layer 117 can be extended along the X-axis direction to be electrically connected to the AC voltage signal line 133 (referring to FIG. 2A), and the second electrode layer 115 can be extended along the Y-axis direction to be electrically connected to the DC voltage signal line 132 (referring to FIG. 2A). However, the invention is not limited thereto.

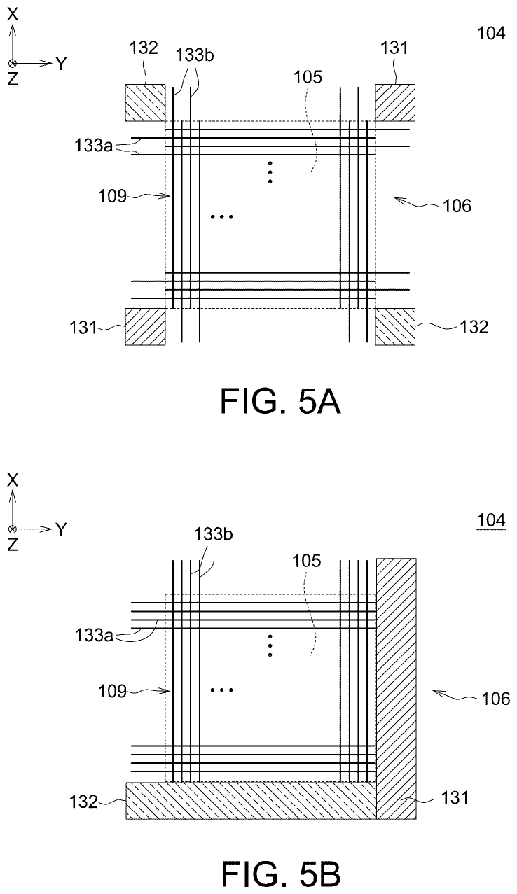

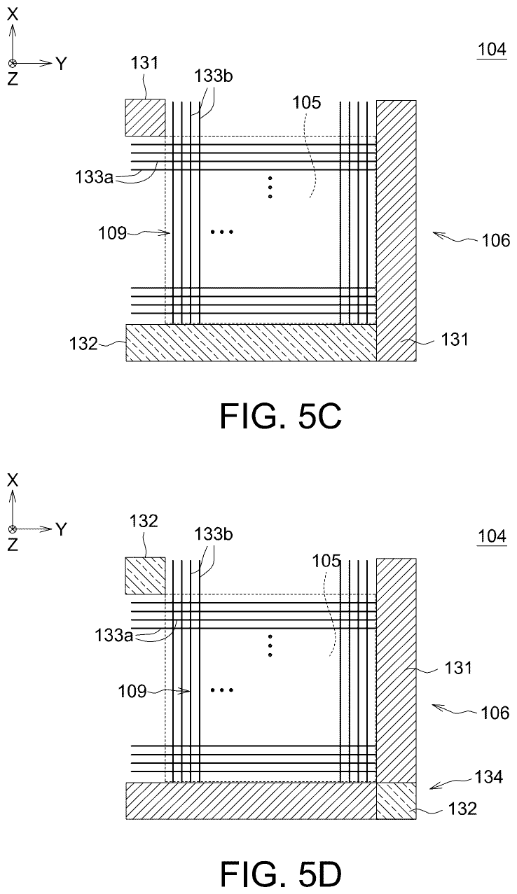

Referring to FIGS. 5A-5D, schematic diagrams of an ultrasonic transducer device 104 according to another embodiment of the invention are respectively shown. In the present embodiment, the active component area 105 includes a plurality of ultrasonic oscillation units 109, two-dimensionally arranged as an MxN matrix, wherein M represents the number of rows and N represents the number of columns. The ultrasonic oscillation units 109 are arranged along an X-axis direction and a Y-axis direction, wherein the Y-axis direction is perpendicular to the X-axis direction. Besides, a plurality of first AC voltage signal lines 133a are arranged on any side or two opposite sides of the active component area 105 along the X-axis direction, and a plurality of second AC voltage signal lines 133b are arranged on any side or two opposite sides of the active component area 105 along the Y-axis direction. The first AC voltage signal lines 133a and the second AC voltage signal lines 133b correspondingly intersect at the two-dimensionally arranged ultrasonic oscillation units 109, so that an alternate current voltage can be correspondingly input to each of the ultrasonic oscillation units 109. The inputs of the first AC voltage signal lines 133a and the second AC voltage signal lines 133b can be one-dimensional or multi-directional.

Besides, the electrical connection area of the ground signal lines 131 and the electrical connection area of the DC voltage signal lines 132 are disposed on any side, two opposite sides, or two adjacent sides of the wiring area 106 along the X-axis direction and the Y-axis direction. For instance, in FIG. 5A, the ground signal lines 131 and the DC voltage signal lines 132 are not adjacent to each other but are disposed at four corners of the wiring area 106. In FIG. 5B, the ground signal lines 131 and the DC voltage signal lines 132 are disposed on two adjacent sides of the wiring area 106, so that the electrical connection area of the ground signal line 131 and the electrical connection area of the DC voltage signal line 132 are respectively located on adjacent sides of the first AC voltage signal line 133a and the second AC voltage signal line 133b. The electrical connection area of the ground signal line 131 and the electrical connection area of the DC voltage signal line 132 can have identical or different levels.

In FIG. 5C, the ground signal line 131 and the DC voltage signal line 132 are disposed on two adjacent sides of the wiring area 106, and the ground signal line 131 are disposed at a corner of the wiring area 106. In FIG. 5D, the ground signal line 131 are disposed on two adjacent sides of the wiring area 106, and the DC voltage signal line 132 are disposed at two corners of the wiring area 106, wherein the projections of the electrical connection area of the ground signal line 131 and the electrical connection area of the DC voltage signal line 132 have an overlapping area 134 in the vertical direction (Z axis) and are electrically isolated from each other.

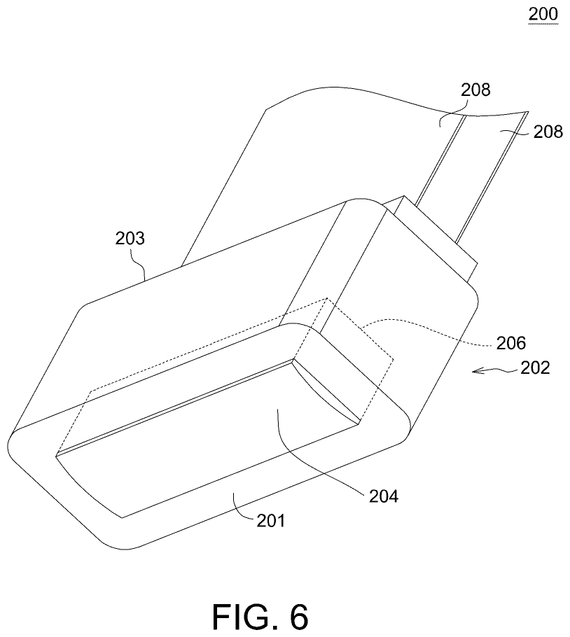

Referring to FIG. 6, a top view of an ultrasonic probe 200 according to an embodiment of the invention is shown. The ultrasonic probe 200 includes a hand-held casing 202, an acoustic lens 204 and an ultrasonic transducer device 206. The hand-held casing 202 has a first end (front end 201) and a second end (back end 203). The acoustic lens 204 is disposed at the first end or the second end. The ultrasonic transducer device 206 is disposed in the hand-held casing 202, and more specifically, on one side of the hand-held casing 202 closer to the acoustic lens 204. In the present embodiment, when the user uses the ultrasonic probe 200 to contact the user's skin, the ultrasonic probe 200 can focus the beams through the acoustic lens 204 at the front end 201 of the hand-held casing 202 then emits or receives ultrasonic signals, and the ultrasonic transducer device 206 can transmit acoustic signals to the ultrasonic device for processing the beams of ultrasonic imaging through the signal transmission line 208. Detailed descriptions of the ultrasonic transducer device 206 can be obtained with reference to the ultrasonic transducer devices 100-104 of above embodiments, and the similarities are not repeated here.

According to each embodiment of the invention, the ground signal line, the DC voltage signal line and the AC voltage signal line are disposed in the wiring area (peripheral circuit area) of the ultrasonic transducer device, so that drive circuit can be integrated to the peripheral circuit of the ultrasonic transducer device, and device volume can be reduced. Since there is no need to connect the peripheral circuit of the ultrasonic transducer device using additional printed circuit board, the need for wiring can be reduced, and the performance of surface-mount type ultrasonic transducer device can be enhanced. Meanwhile, as different methods of electrical connection are provided in response to various wiring requirements of the circuit design of the ultrasonic transducer device, device volume can be further reduced, and miniaturization and portability of the device can be achieved.

While the invention has been described by way of example and in terms of the preferred embodiment(s), it is to be understood that the invention is not limited thereto. Based on the technical features embodiments of the present invention, a person ordinarily skilled in the art will be able to make various modifications and similar arrangements and procedures without breaching the spirit and scope of protection of the invention. Therefore, the scope of protection of the present invention should be accorded with what is defined in the appended claims.

What is claimed is:

1. An ultrasonic transducer device, comprising:
a substrate, comprising an active component area and a wiring area located around the active component area;
an ultrasonic oscillation unit disposed in the active component area, wherein the ultrasonic oscillation unit comprises a first electrode layer, a second electrode layer, a third electrode layer and a cavity; the first electrode layer is located on a first side of the cavity, the second electrode layer and the third electrode layer are located on a second side of the cavity, and the first side is opposite to the second side;
a ground signal line disposed in the wiring area and electrically connected to the first electrode layer;
a direct current (DC) voltage signal line disposed in the wiring area and electrically connected to the second electrode layer; and
an alternate current (AC) voltage signal line disposed in the wiring area and electrically connected to the third electrode layer,
wherein, the ground signal line and the DC voltage signal line have electrical connection areas of different levels in the wiring area.

2. The ultrasonic transducer device according to claim 1, wherein the active component area has a first side connection area and a second side connection area; the ground signal line and the DC voltage signal line are disposed in the first side connection area and are electrically connected to at least a corresponding part of the ultrasonic oscillation units, the AC voltage signal line is disposed in the second side connection area and is electrically connected to at least another corresponding part of the ultrasonic oscillation units, and the first side connection area is different from the second side connection area.

3. The ultrasonic transducer device according to claim 1, wherein a plurality of the ultrasonic oscillation units one-dimensionally arranged in the active component area to form a transducer unit; and a plurality of the transducer units are arranged in parallel along a first direction, and the electrical connection area of the ground signal line and the electrical connection area of the DC voltage signal line are disposed on any side or two opposite sides of the wiring area along the first direction.

4. The ultrasonic transducer device according to claim 3, wherein the AC voltage signal line is disposed on any side or two opposite sides of the wiring area along a second direction perpendicular to the first direction.

5. The ultrasonic transducer device according to claim 4, wherein the AC voltage signal line is disposed on a first side of the wiring area along the second direction, the ground signal line further comprises an electrical connection area disposed on a second side of the wiring area along the second direction, and the first side is opposite to the second side.

6. The ultrasonic transducer device according to claim 3, wherein the electrical connection area of the DC voltage signal line has a first level, the electrical connection area of the ground signal line has a second level, and the second level is higher than the first level.

7. The ultrasonic transducer device according to claim 6, wherein the electrical connection area of the ground signal line crosses over the electrical connection area of the DC voltage signal line in a second direction perpendicular to the first direction.

8. The ultrasonic transducer device according to claim 3, wherein the electrical connection area of the ground signal line has a first level, the electrical connection area of the DC voltage signal line has a second level, and the second level is higher than the first level.

9. The ultrasonic transducer device according to claim 8, wherein the electrical connection area of the DC voltage signal line crosses over the electrical connection area of the ground signal line in a second direction perpendicular to the first direction.

10. The ultrasonic transducer device according to claim 1, wherein the second electrode layer and the third electrode layer have an electrical connection area of different levels in the active component area.

11. The ultrasonic transducer device according to claim 1, wherein the second electrode layer and the third electrode layer have electrical connection areas of identical level in the active component area.

12. The ultrasonic transducer device according to claim 1, wherein the ultrasonic oscillation units are two-dimensionally arranged along a first direction and a second direction perpendicular to the first direction; the electrical connection areas of the ground signal line and the DC voltage signal line are disposed on any side or two opposite sides of the wiring area along the first direction and the second direction.

13. The ultrasonic transducer device according to claim 12, wherein the AC voltage signal line is disposed on any side or two opposite sides of the wiring area along the first direction and the second direction; the electrical connection areas of the ground signal line and the DC voltage signal line are respectively located on an adjacent side of the AC voltage signal line.

14. The ultrasonic transducer device according to claim 1, wherein the second electrode layer, relative to the first electrode layer, is driven by a direct current voltage to recess towards the cavity, making the first electrode layer closer to the second electrode layer.

15. The ultrasonic transducer device according to claim 1, wherein the third electrode layer, relative to the first electrode layer, is driven by an alternate current voltage or an external acoustic pressure to generate vibrations.

16. The ultrasonic transducer device according to claim 12, wherein the third electrode layer, relative to the first electrode layer, is driven by an alternate current voltage or an external acoustic pressure to generate vibrations.

17. An ultrasonic probe, comprising:
a hand-held casing, wherein the hand-held casing has a first end and a second end; and
an ultrasonic transducer device disposed at the first end or the second end of the hand-held casing, wherein the ultrasonic transducer device comprises:
a substrate, comprising an active component area and a wiring area located around the active component area;
an ultrasonic oscillation unit disposed on the active component area, wherein the ultrasonic oscillation unit comprises a first electrode layer, a second electrode layer, a third electrode layer and a cavity; the first electrode layer is located on a first side of the cavity, the second electrode layer and the third electrode layer are located on a second side of the cavity, and the first side is opposite to the second side;
a ground signal line disposed in the wiring area and electrically connected to the first electrode layer;
a DC voltage signal line disposed in the wiring area and electrically connected to the second electrode layer; and
an AC voltage signal line disposed in the wiring area and electrically connected to the third electrode layer,
wherein the second electrode layer and the third electrode layer have electrical connection areas of different levels in the active component area.

18. The ultrasonic probe according to claim 17, wherein the second electrode layer, relative to the first electrode layer, is driven by a direct current voltage to recess towards the cavity, making the first electrode layer closer to the second electrode layer.

19. An ultrasonic probe, comprising:
a hand-held casing, wherein the hand-held casing has a first end and a second end; and
an ultrasonic transducer device disposed on the first end or the second end of the hand-held casing, wherein the ultrasonic transducer device comprises:
a substrate, comprising an active component area and a wiring area located around the active component area;
an ultrasonic oscillation unit disposed in the active component area, wherein the ultrasonic oscillation unit comprises a first electrode layer, a second electrode layer, a third electrode layer and a cavity; the first electrode layer is located on a first side of the cavity, the second electrode layer and the third electrode layer are located on a second side of the cavity, and the first side is opposite to the second side;
a ground signal line disposed in the wiring area and electrically connected to the first electrode layer;
a DC voltage signal line disposed in the wiring area and electrically connected to the second electrode layer; and
an AC voltage signal line disposed in the wiring area and electrically connected to the third electrode layer;
wherein the second electrode layer and the third electrode layer have electrical connection areas of identical level in the active component area.

20. The ultrasonic probe according to claim 19, wherein the second electrode layer, relative to the first electrode layer, is driven by a direct current voltage to recess towards the cavity, making the first electrode layer closer to the second electrode layer.
